(19) United States
(12) Patent Application Publication
(10) Pub. No.: US 2024/0141314 A1
(43) Pub. Date: May 2, 2024

(54) INFORMATION PROCESSING DEVICE, DETACHMENT ASSISTANCE SYSTEM, NON-TRANSITORY STORAGE MEDIUM, INFORMATION PROCESSING METHOD, AND CELL DETACHMENT METHOD

(71) Applicant: CANON KABUSHIKI KAISHA, Tokyo (JP)

(72) Inventors: KENJIRO TAKEMURA, Tokyo (JP); YUTA KURASHINA, Tokyo (JP); CHIKAHIRO IMASHIRO, Tokyo (JP); KAZUNORI NOGUCHI, Ibaraki (JP); MASASHI HIROSE, Tokyo (JP); KATSUHISA YAMAZAKI, Shizuoka (JP); KEIICHIRO TSUBAKI, Tokyo (JP); SUGURU WATANABE, Kanagawa (JP); AKIRA SUGIYAMA, Kanagawa (JP); KENICHI KAKU, Shizuoka (JP); RYUICHI OTSU, Tokyo (JP); YUKARI NAKASHOJI, Saitama (JP); TAKAAKI FURUI, Tokyo (JP); TATSUO FURUTA, Tokyo (JP); HITOMI TOKUTAKE, Kanagawa (JP)

(21) Appl. No.: 18/488,168

(22) Filed: Oct. 17, 2023

(30) Foreign Application Priority Data
Oct. 26, 2022 (JP) 2022-171678

(51) Int. Cl. C12N 13/00; C12M 1/24; C12M 1/34; C12M 1/36; C12M 1/42

(52) U.S. Cl. CPC C12N 13/00; C12M 23/08; C12M 35/04; C12M 41/46; C12M 41/48

(57) ABSTRACT

The information processing device includes: a cultivation information acquisition unit configured to acquire cultivation information of a cell; and a recommended condition setting unit configured to set a recommended condition for detaching the cell from a to be-processed vessel in which the cell is cultured, the cultivation information including information about the to-be-processed vessel, the recommended condition being a condition for detaching the cell by applying vibration to the to-be-processed vessel, the recommended condition setting unit being configured to set the recommended condition based on the cultivation information, with use of information concerning an association relationship between information about a cultivation condition and information about a detachment condition, the information about the cultivation condition including information about a culture vessel, the information about the detachment condition including information about a condition for applying vibration to the culture vessel.

### BACKGROUND OF THE INVENTION

### Field of the Invention

The present invention relates to an information processing device, a detachment assistance system, non-transitory storage medium, an information processing method, and a cell detachment method.

### Description of the Related Art

Cell culture for culturing a cell to be used in regenerative medicine and a cell to be used in manufacture of a bio-pharmaceutical or the like is actively practiced. Cell culture is roughly divided into adherent culture and floating culture. Adherent culture, in particular, is suitable for various cells and, accordingly, is widely used.

In order to collect cells cultured by adherent culture, at a high detachment rate with damage to the cells suppressed, diverse examinations are being done on detachment conditions.

Methods of detaching a cell from a culture vessel include one in which the cell is detached by applying vibration to the culture vessel.

In International Publication No. WO2016/047368, there is proposed a device which detaches cells by obtaining, through structural analysis, a natural vibration frequency, which changes depending on a state inside a culture vessel, the number of layers or stages of the culture vessel, and other factors, and sweeping an applied vibration frequency based on the natural vibration frequency.

Examinations on devices that provide assistance in cell culture are also being done and, in International Publication No. WO2020/039683, there is proposed a support device which derives predicted culture result data by predicting culture result data of a production culture step from initial performance data for analysis and provisional cultivation condition data.

According to examinations made by the inventors of the present invention, a cell dissociation device as described in International Publication No. WO2016/047368 requires an advance examination on a cell dissociation condition optimum for a cultivation condition including a type of a culture vessel used, in order to dissociate cells stably and effectively.

The support device as described in International Publication No. WO2020/039683 is unsuitable for improving efficiency of determination of a cell detachment condition in, for example, the device as described in International Publication No. WO2016/047368.

### SUMMARY OF THE INVENTION

It is accordingly an object of the present invention to provide an information processing device usable in a method of detaching a cell by applying vibration to a culture vessel, to set a cell detachment condition appropriate for a cultivation condition including a type of culture vessel used.

Another object of the present invention is to provide a detachment assistance system, an information processing method, and a cell detachment method that use the above-mentioned information processing device.

Still another object of the present invention is to provide a non-transitory storage medium having stored thereon an information processing program that is used in the above-mentioned information processing device.

The above-mentioned objects are achieved by the present invention described below.

That is, according to one aspect of the present invention, there is provided an information processing device including: a cultivation information acquisition unit configured to acquire cultivation information of a cell; and a recommended condition setting unit configured to set a recommended condition for detaching the cell from a to-be-processed vessel in which the cell is cultured, the cultivation information including information about the to-be-processed vessel, the recommended condition being a condition for detaching the cell by applying vibration to the to-be-processed vessel, the recommended condition setting unit being configured to set the recommended condition based on the cultivation information, with use of information concerning an association relationship between information about a cultivation condition and information about a detachment condition, the information about the cultivation condition including information about a culture vessel, the information about the detachment condition including information about a condition for applying vibration to the culture vessel.

Further, according to one aspect of the present invention, there is provided a detachment assistance system including: a measurement device configured to execute measurement for acquisition of the cultivation information; and the above-mentioned information processing device, wherein the measurement device includes a vessel measurement device configured to execute measurement for acquisition of the information about the to-be-processed vessel.

Further, according to one aspect of the present invention, there is provided a non-transitory storage medium having stored thereon an information processing program for causing a computer to: acquire cultivation information of a cell; and set a recommended condition for detaching the cell from a to-be-processed vessel in which the cell is cultured, the setting being executed based on the cultivation information, with use of information concerning an association relationship between information about a cultivation condition and information about a detachment condition, the cultivation information including information about the to-be-processed vessel, the recommended condition being a condition for detaching the cell by applying vibration to the to-be-processed vessel, the information about the cultivation condition including information about a culture vessel, the information about the detachment condition including information about a condition for applying vibration to the culture vessel.

Further, according to one aspect of the present invention, there is provided an information processing method including: a cultivation information acquisition step of acquiring cultivation information of a cell; and a recommended condition setting step of setting a recommended condition for detaching the cell from a to-be-processed vessel in which the cell is cultured, the cultivation information including information about the to-be-processed vessel, the recommended condition being a condition for detaching the cell by applying vibration to the to-be-processed vessel, the recommended condition setting step including setting the recommended condition based on the cultivation information, with use of information concerning an association relationship between information about a cultivation condition and information about a detachment condition, the information about the cultivation condition including information about a culture vessel, the information about the detachment condition including information about a condition for applying vibration to the culture vessel.

Further, according to one aspect of the present invention, there is provided a cell detachment method including: a recommended condition acquisition step of acquiring, with use of the above-mentioned information processing device, the recommended condition; a processing condition setting step of setting, based on the recommended condition, a processing condition for detachment a cell from the to-be-processed vessel; and a detachment step of detaching the cell from the to-be-processed vessel, based on the processing condition.

Further features of the present invention will become apparent from the following description of exemplary embodiments with reference to the attached drawings.

### BRIEF DESCRIPTION OF THE DRAWINGS

FIG. 1 is a function block diagram of an information processing device according to a first embodiment of the present invention.

FIG. 2 is a flow chart for illustrating an outline of processing to be executed by the information processing device according to the first embodiment of the present invention.

FIG. 3 is a block diagram for illustrating a hardware configuration of the information processing device according to the first embodiment of the present invention.

FIG. 4A, FIG. 4B, FIG. 4C, FIG. 4D, FIG. 4E, and FIG. 4F are schematic diagrams for illustrating examples of a culture vessel.

FIG. 5 is a schematic table for showing an example of a case in which information concerning an association relationship between information about cultivation conditions and information about detachment conditions is held as table information.

FIG. 6 is a schematic diagram for illustrating an operation example of a display unit included in the information processing device according to the first embodiment of the present invention.

FIG. 7 is a function block diagram of a detachment assistance system according to a second embodiment of the present invention.

FIG. 8 is a flow chart for illustrating an outline of a cell detaching method that uses the detachment assistance system according to the second embodiment of the present invention.

### DESCRIPTION OF THE EMBODIMENTS

Exemplary embodiments of the present invention are now described with reference to the drawings. Like elements or corresponding elements are denoted by the same reference symbols in the drawings, and description thereof may be omitted or simplified.

### First Embodiment

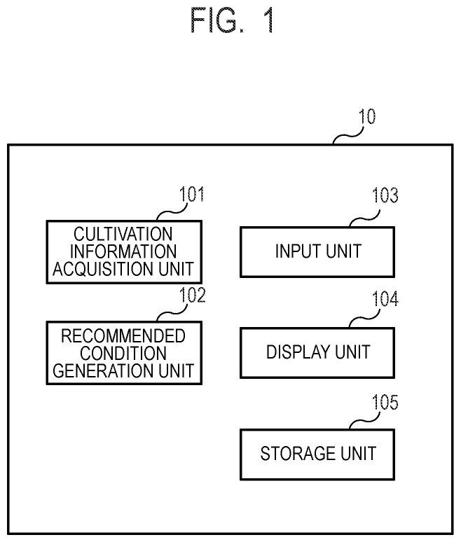

FIG. 1 is a block diagram of an information processing device 10 according to a first embodiment of the present invention.

As illustrated in FIG. 1, the information processing device 10 according to the present embodiment includes a cultivation information acquisition unit 101, a recommended condition setting unit 102, an input unit 103, a display unit 104, and a storage unit 105. The information processing device 10 according to the present embodiment may include a display control unit (not shown).

The information processing device 10 according to the present embodiment is used in a method of detaching a cell by applying vibration to a culture vessel, in order to set, based on cultivation information, a recommended condition usable for efficient setting of an optimum condition under which the cell is detached from the culture vessel. The cell to be detached here can be any of a single cell, an aggregate in which a plurality of cells clump together, and cells cultured in a shape of a sheet (a cell sheet) (the same applies to the following).

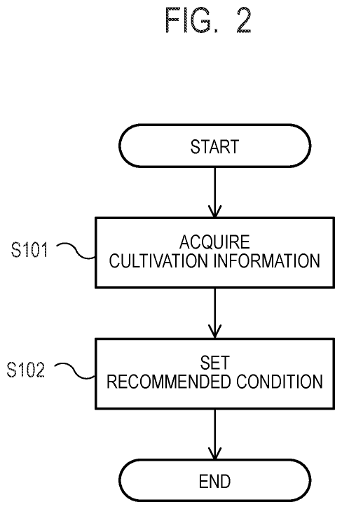

FIG. 2 is a flow chart for illustrating an outline of processing to be executed by the information processing device 10. In Step S101, which is a cultivation information acquisition step, the cultivation information acquisition unit 101 acquires cultivation information of a cell based on input from the input unit 103. The cultivation information acquired by the cultivation information acquisition unit 101 includes information about a to-be-processed vessel from which the cell is to be detached. The information acquired by the cultivation information acquisition unit 101 is transmitted to the recommended condition setting unit 102. Prior to, or at the same time as, the transmission to the recommended condition setting unit 102, the acquired information may be stored in the storage unit 105.

In Step S102, which is a recommended condition setting step, the recommended condition setting unit 102 sets, based on the cultivation information acquired by the cultivation information acquisition unit 101, a recommended condition for detaching the cell from the to-be-processed vessel in which the cell has been cultured. In the present embodiment, the recommended condition setting unit 102 sets the recommended condition with the use of information stored in the storage unit 105 as information concerning an association relationship between information about cultivation conditions and information about detachment conditions. Here, the information about cultivation conditions includes information about a culture vessel, and the information about detachment conditions includes information about a condition under which vibration is applied to the culture vessel. The recommended condition setting unit 102 may be designed so as to use one piece of “information concerning the association relationship between the information about cultivation conditions and the information about detachment conditions” selected from a plurality of pieces of “information concerning the association relationship between the information about cultivation conditions and the information about detachment conditions” different from one another. In this case, the one piece of “information concerning the association relationship between the information about cultivation conditions and the information about detachment conditions” may be selected by a user each time, or may be selected automatically based on a criterion determined in advance.

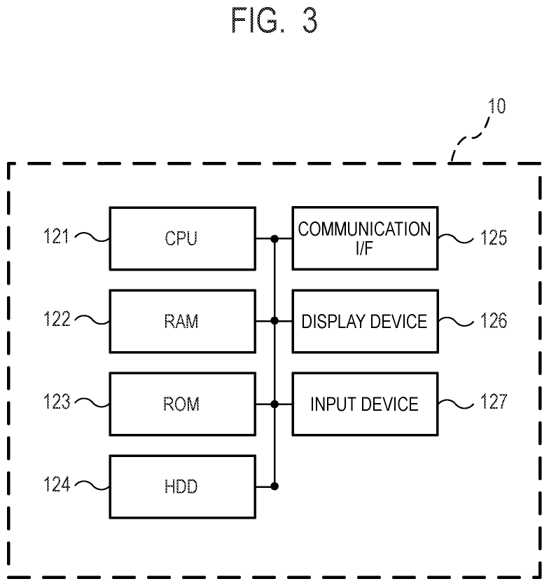

FIG. 3 is a block diagram for illustrating a hardware configuration of the information processing device 10 according to the present embodiment. The information processing device 10 includes a Central Processing Unit (CPU) 121, a Random Access Memory (RAM) 122, a Read Only Memory (ROM) 123, a hard disk drive (HDD) 124, a communication OF 125, a display device 126, and an input device 127. These units are interconnected via a bus or the like.

The CPU 121 is a processor that reads programs stored in the ROM 123 and the HDD 124 into the RAM 122 and executes the programs, and performs calculation processing, control of each unit of the information processing device 10, and the like. The processing performed by the CPU 121 may include acquisition of information of cultivation conditions, setting of a recommended condition using correspondence information between information of cultivation conditions and information of detachment conditions, and the like.

The RAM 122 is a volatile storage medium and functions as a work memory when the CPU 121 executes a program. The ROM 123 is a non-volatile storage medium, and stores firmware and the like necessary for the operation of the information processing device 10. The HDD 124 is a non-volatile storage medium, and stores information acquired by the cultivation information acquisition unit 101, correspondence between information of cultivation conditions and information of detachment conditions which is used by the recommended condition setting unit 102, programs used for acquisition of cultivation information or setting of a recommended condition, and the like.

The communication OF 125 is a communication device based on a standard such as Wi-Fi (registered trademark), Ethernet (registered trademark), or Bluetooth (registered trademark). The communication OF 125 is used for communication with a measurement device and a detachment device which will be described below, and another computer, or the like.

The input device 127 is a device for inputting information to the information processing device 10, and is typically a user interface for a user to operate the information processing device 10. Examples of the input device 127 include a keyboard, buttons, a mouse, and a touch panel.

The display device 126 is a device in which the information processing device 10 outputs information to the outside, and is typically a user interface for presenting information to the user. Examples of the display device 126 include a display and a speaker.

Note that the configuration of the information processing device 10 described above is merely an example, and can be changed as appropriate. Examples of processors that can be mounted on the information processing device 10 include a Graphics Processing Unit (GPU), an Application Specific Integrated Circuit (ASIC), and a Field Programmed Gate Array (FPGA) in addition to the CPU 121. A plurality of processors may be provided, or a plurality of processors may perform processing in a distributed manner. The function of storing information such as image data in the HDD 124 may be provided not in the information processing device 10 but in another data server. The HDD 124 may be a storage medium such as an optical disk, a magneto-optical disk, or a solid-state drive (SSD).

The CPU 121 executes a program to perform predetermined calculation processing. The CPU 121 controls each unit in the information processing device 10 by executing a program. Through these processes, the CPU 121 realizes the functions of the cultivation information acquisition unit 101 and the recommended condition setting unit 102.

The hardware configuration illustrated in FIG. 3 is an example, and devices other than the illustrated devices may be added, or part of the illustrated devices may be omitted as long as the functions of the information processing device 10 according to the present embodiment are realized. In addition, part of the devices may be substituted with another device having the same function. Further, part of the functions may be provided by another device via a network, and the functions for implementing the embodiments may be shared and implemented by a plurality of devices. For example, the HDD 124 may be substituted with a solid state drive (SSD) using a semiconductor element such as a flash memory.

The recommended condition setting unit 102 may automatically set the recommended condition based on the information acquired by the cultivation information acquisition unit 101, or may set the recommended condition based on input from the input unit 103.

The recommended condition set by the recommended condition setting unit 102 may be stored in the storage unit 105.

The display unit 104 can display the information acquired by the cultivation information acquisition unit 101 and the recommended condition set by the recommended condition setting unit 102. In a case in which a user of the information processing device 10 is not required to check the information acquired by the cultivation information acquisition unit 101 and the recommended condition set by the recommended condition setting unit 102, the information processing device 10 may omit the display unit 104.

The information about the culture vessel in the above-mentioned association relationship referred to by the recommended condition setting unit 102 can include at least one type of information selected from the group consisting of the following four types of information. That is, the four types of information are information about a type of the culture vessel, information about a shape of the culture vessel, information about a chemical property of the culture vessel, and information about a physical property of the culture vessel.

The inventors of the present invention have found out that culture vessels are commercially available from a plurality of companies, and that culture vessels similar to each other in form sometimes differ from each other in cell detachment property when the culture vessels are different in terms of manufacturing company, sales company, or part number. A reason for the varied detachment property from one culture vessel to another culture vessel is considered to be a difference in at least one of shapes, chemical properties, and physical properties of the culture vessels regardless of the similarity. For example, in a case of dishes purposed for culture of a hydrophilic cell, culture vessels similar to each other may differ in thickness of a bottom surface portion or a side surface portion, presence or absence of a convex portion in the side surface portion, or a shape, a material, or the like of the convex portion. According to the examination of the inventors of the present invention, a frequency of the vibration is preferred to be higher when the bottom surface portion increases in density, and a higher vibration intensity tends to be preferred when the side surface portion is thicker.

In International Publication No. WO2016/047368, there is no mention about the cell dissociation device varying in detachment property depending on the manufacturing company and part number of the culture vessel.

From the above-mentioned examination, the information about the type of the culture vessel may include at least one piece of information selected from the group consisting of information about a manufacturing company of the culture vessel, information about a sales company of the culture vessel, and information about a part number of the culture vessel. The information about the type of the culture vessel is not information directly related to characteristics of the culture vessel. However, in some cases, the culture vessel itself or features of the culture vessel such as a material and a shape can be identified based on the information about the type of the culture vessel. An optimum condition for detachment can accordingly be set based on the culture vessel or features of the culture vessel identified from the information about the type of the culture vessel.

Further, the information about the shape of the culture vessel may include at least one type of information selected from the group consisting of the following four types of information. The four types of information are information about a form of the culture vessel, information about a thickness of the culture vessel, information about a curvature of the bottom surface portion of the culture vessel, and information about a flatness and smoothness of an inner surface of the culture vessel.

Forms of the culture vessel include typical shapes of a flask, a dish, a well, and the like, and a cell can effectively be detached by applying vibration under a condition suitable for one of those shapes. Culture vessel types may include a dish type, and the information about the shape of the culture vessel may include information about presence or absence of a convex portion provided in a bottom surface edge portion of the dish. When there is a convex portion provided in the bottom surface edge portion of the dish, the information about the shape of the culture vessel may include information about a shape of the convex portion.

The thickness of the culture vessel and the curvature of the bottom surface portion affect transmission of the vibration in particular, and the flatness and smoothness of the inner surface of the culture vessel affect an adherence property of the cell. Accordingly, the cell can effectively be detached by applying vibration under a condition that takes those into consideration.

In addition, the information about the chemical property of the culture vessel may include at least one piece of information selected from the group consisting of information about a material forming the culture vessel, and information about chemical modification of the inner surface of the culture vessel.

Examples of the material of the culture vessel include glass, resin, and stainless steel. Of those, polystyrene vessels are widely used. One polystyrene vessel and another polystyrene vessel may differ in characteristics such as density and Young's modulus. Those differences in material forming the culture vessel affect transmission of the vibration and the adherence property of the cell. For chemical modification of the inner surface of the culture vessel, fibronectin, collagen, lysine, laminin, and the like are commonly used. Thermo-responsive chemical modification and photo-responsive chemical modification are used in some cases. Those can affect the adherence property of the cell. Accordingly, the cell can effectively be detached by applying vibration under a condition that takes those into consideration.

Further, the information about the physical property of the culture vessel may include at least one type of information selected from the group consisting of the following four types of information. The four types of information are information about hydrophilic property of the inner surface of the culture vessel, information about a contact angle of the inner surface of the culture vessel, information about a surface charge of the inner surface of the culture vessel, and information about a natural vibration frequency of the culture vessel.

The hydrophilic property of the inner surface of the culture vessel, the contact angle of the inner surface of the culture vessel, and the surface charge of the inner surface of the culture vessel each affect the adherence property of the cell in particular. The natural vibration frequency of the culture vessel affects transmission of the vibration to the culture vessel. Accordingly, the cell can effectively be detached by applying vibration under a condition that takes those into consideration.

The natural vibration frequency of the culture vessel can be a vibration frequency of a vibration that has been applied at a time when the vibration frequency of the culture vessel reaches a local maximum during application of a vibration in a certain range of vibration frequencies to the culture vessel. The vibration frequency of an applied vibration at which the vibration frequency of the culture vessel reaches the local maximum may be calculated by simulation. When the vibration frequency of the culture vessel has a plurality of local maximum values, a maximum value out of the plurality of local maximum values can be selected. A local maximum value of the vibration frequency of the culture vessel is measurable with a laser doppler or the like.

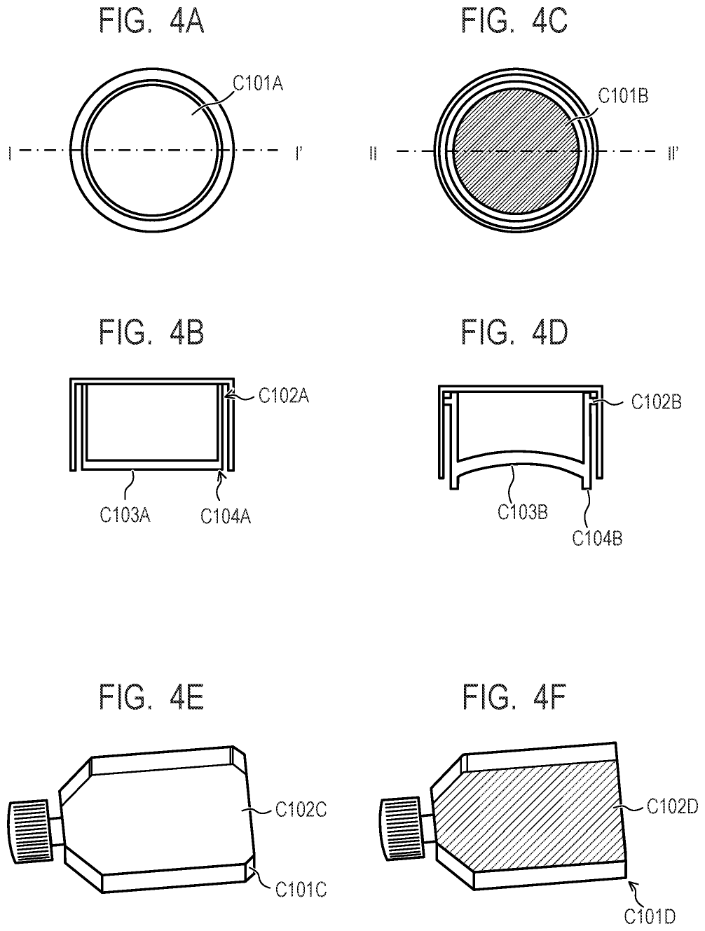

FIG. 4A to FIG. 4F are schematic diagrams for illustrating examples of the culture vessel. FIG. 4A is a top view of a culture vessel A. FIG. 4B is a schematic diagram of a vertical sectional view taken along the line I-I′ of FIG. 4A.

FIG. 4C is a top view of a culture vessel B, which is another example of the culture vessel. FIG. 4D is a schematic diagram of a vertical sectional view taken along the line II-IF of FIG. 4C. FIG. 4E is a perspective view of a culture vessel C. FIG. 4F is a perspective view of a culture vessel D.

The culture vessel A and the culture vessel B differ from each other in chemical modification and water contact angles of bottom surfaces of the vessels, shapes of circumferential wall surfaces C102A and C102B, thicknesses and curvatures of bottom surfaces C103A and C103B, and shapes of bottom surface edge portions C104A and C104B. The culture vessel A has no convex portions in the wall surface C102A and the bottom surface edge portions C104A. The culture vessel B, on the other hand, has convex portions in the wall surface C102B and the bottom surface edge portion C104B. The culture vessel C and the culture vessel D differ from each other in chemical modification and water contact angles of bottom surfaces C102C and C102D, and shapes of corners C101C and C101D.

As illustrated in the examples of the culture vessels A to D described above, features that may affect the adherence property of the cell and transmission of the vibration vary from one culture vessel to another culture vessel, and the cell can effectively be detached by applying vibration suited to those features.

The culture vessel from which the information about the culture vessel is acquired is preferred to include a versatile culture vessel, and may include at least one selected from the group consisting of a flask, a dish, and a well.

In addition to the information about the culture vessel, the information about cultivation conditions may further include at least one type of information selected from the group consisting of the following five types of information. The five types of information are information about a cell, information about a cell semination density, information about a culture fluid, information about a cultivation period, and information about a culture environment. Here, the information about cells may include information about cell species. The information about the culture fluid may include a quantity of the culture fluid, a composition of the culture fluid, and a replacement condition of the culture fluid. The information about the culture environment may include temperature, humidity, illuminance, a composition of a gas supplied to the culture vessel, a rate at which the gas is supplied to the culture vessel, a stirring velocity of the culture fluid, and a condition for shaking the culture vessel. Those can affect the adherence property of the cell in particular. Accordingly, the cell can effectively be detached by applying vibration under a condition that takes those into consideration.

In the present embodiment, as a matter of course, the information about cultivation conditions in the association relationship described above which is referred to by the recommended condition setting unit 102 inevitably includes information of the same category as a category of at least some of the cultivation information acquired by the cultivation information acquisition unit 101. That is, when the cultivation information acquired by the cultivation information acquisition unit 101 is only information about the thickness of the culture vessel, for example, the information about cultivation conditions in the association relationship described above inevitably includes information about the thickness of the culture vessel. This enables the recommended condition setting unit 102 to set the recommended condition based on the cultivation information acquired by the cultivation information acquisition unit 101, with the use of the association relationship described above.

Meanwhile, the information about conditions under which vibration is applied to the culture vessel may include at least one type of information selected from the group consisting of the following three types of information. The three types of information are information about an intensity of the vibration to be applied to the culture vessel, information about a frequency of the vibration to be applied to the culture vessel, and information about a time for which the vibration is applied to the culture vessel. The cell can effectively be detached by adjusting the intensity and frequency of the vibration to be applied to the culture vessel and the time for which the vibration is applied to the culture vessel, so as to suit the cultivation condition of the cell. In particular, when the vibration applied to the culture vessel is vibration caused by application of an ultrasonic wave that is generated by an ultrasonic transducer, the information about the intensity of the vibration to be applied to the culture vessel may include information about a voltage to be applied to the ultrasonic transducer.

The information about the detachment condition may further include, in addition to the information about the conditions under which the vibration is applied, at least one piece of information selected from the group consisting of information about a detachment fluid and information about an acoustic matching material. The cell can effectively be detached by adjusting the type and quantity of the detachment fluid added to the culture vessel when vibration is applied, the type of the acoustic matching material used when vibration is applied, and the like, so as to suit the cultivation condition of the cell.

The information about the detachment condition is typically information obtained based on information about a detachment result. The information about the detachment result here may include at least one piece of information selected from the group consisting of information about a detachment rate of the cell, information about a survival rate of the detached cell, information about a growth rate of the detached cell, and information about functionality of the detached cell. The information about the detachment result here corresponds to demanded detachment performance which varies depending on the use of the detached cell. That is, when a cultured cell is to be collected at as high a ratio as possible, for example, the information about detachment conditions may include information acquired based on the information about the detachment rate of the cell.

The information concerning the association relationship between the information about cultivation conditions and the information about detachment conditions which is referred to by the recommended condition setting unit 102 may be, for example, table information.

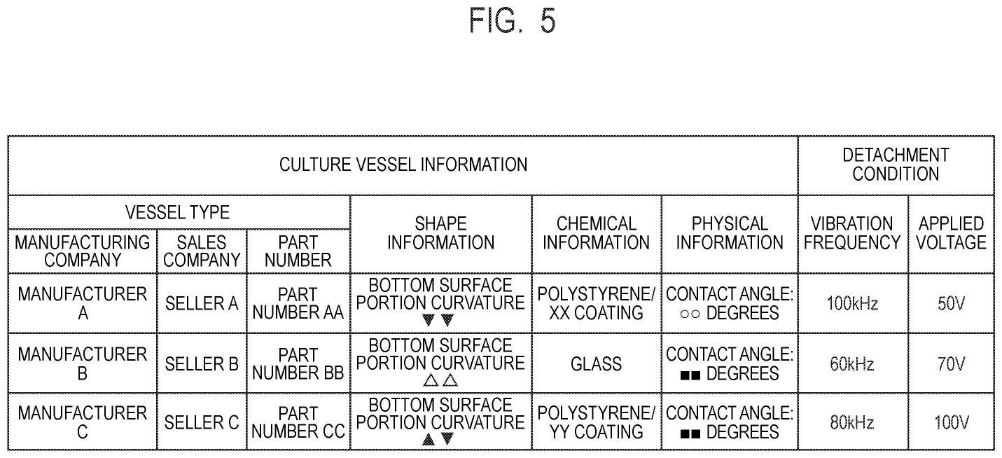

FIG. 5 is a schematic table for showing an example of a case in which the information concerning the association relationship between the information about cultivation conditions and the information about detachment conditions is held as table information.

In FIG. 5, a case in which the information about cultivation conditions includes only information about the culture vessel is illustrated. The information about the culture vessel in the example illustrated in FIG. 5 includes the information about the type of the culture vessel, the information about the shape of the culture vessel, the information about the chemical property of the culture vessel, and the information about the physical property of the culture vessel. Further, the information about the type of the culture vessel includes the information about the manufacturing company of the culture vessel, the information about the sales company of the culture vessel, and the information about the part number of the culture vessel. Those pieces of information about cultivation conditions are associated with the frequency of vibration applied to the culture vessel and a voltage applied to the ultrasonic transducer which are included as the information about detachment conditions.

The association relationship illustrated in FIG. 5 is merely an example, and the information about cultivation conditions and the information about detachment conditions to be included in the table information are not limited to those illustrated in FIG. 5. The association relationship between the information about cultivation conditions and the information about detachment conditions which is referred to by the recommended condition setting unit 102 may be expressed in a mode other than a table mode.

When there are a plurality of reference items based on which the recommended condition setting unit 102 sets the recommended condition, the association relationship between the information about cultivation conditions and the information about detachment conditions which is referred to by the recommended condition setting unit 102 is not limited to a one-to-one association. That is, when the information about detachment conditions includes a plurality of items, for example, a plurality of detachment conditions that are varied combinations of the plurality of included items may be associated with a single cultivation condition. In this case, the recommended condition setting unit 102 may set the recommended condition in order of priority determined for the plurality of reference items in advance.

For example, the following case is considered: candidate detachment conditions for detachment of a cell cultured in a specific vessel are a detachment condition A under which detachment finishes in a short time but the cell survival rate is slightly low, and a detachment condition B under which detachment takes time but the cell can be detached at a high survival rate. In this case, a rule that gives priority to the time required for detachment over the cell survival rate may be placed in advance, and the detachment condition A may be set as the recommended condition. Alternatively, a rule that gives priority to the cell survival rate over the time required for detachment may be placed in advance, and the detachment condition B may be set as the recommended condition.

Each of the items related to the order of priority is not required to be associated with one of the plurality of categories included in the information about detachment conditions on a one-to-one basis and, for example, one index derived from a plurality of categories may be included in the order of priority. To give a specific example, an index with respect to energy required for detachment may be created from the information about the intensity of the vibration to be applied to the culture vessel and the information about the time for which the vibration is applied to the culture vessel, and newly included in the order of priority so that a condition that keeps the energy small is set as the recommended condition.

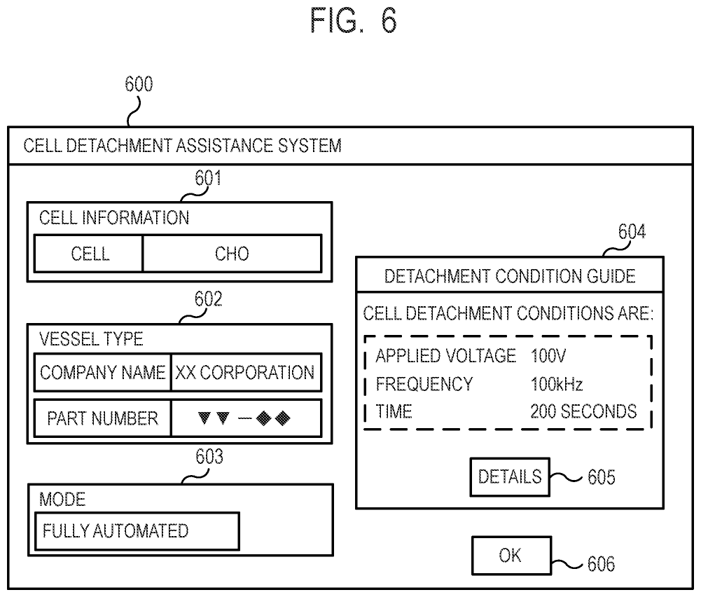

FIG. 6 is a schematic diagram for illustrating an operation example of the display unit 104 included in the information processing device 10 according to the present embodiment.

A display area of the display unit 104 displays a general display window 600 in which information about operation of the information processing device 10 according to the present embodiment is displayed in an integrated manner. The general display window 600 includes a cell information input window 601, a vessel type input window 602, a mode setting window 603, a condition display window 604, and an approval button 606.

The cell information input window 601 is a window for inputting the type of a cell to be detached. The vessel type input window 602 is a window for inputting, as the type of the vessel, a manufacturing company name and a part number. The mode setting window 603 is a window for selecting a mode that corresponds to the order of priority used as a criterion when the recommended condition setting unit 102 sets the recommended condition.

Examples of the mode selected in the mode setting window 603 include a fully automated mode, an applied voltage prioritized mode, a time prioritized mode, an energy saving mode, and a reference mode.

Here, the fully automated mode is a mode for setting, as the recommended condition, a detachment condition that is optimum in accordance with a given criterion set in advance. The applied voltage prioritized mode and the time prioritized mode are modes that place priority to a detachment condition under which the voltage applied to the ultrasonic transducer is low and a detachment condition under which the time required for detachment is short, respectively, as the recommended condition to be set. The energy saving mode is a mode for setting, as the recommended condition, a detachment condition that keeps a total energy required for detachment of the cell small. The reference mode is a mode in which settings in a given criterion set in advance are referred to and, after the settings are changed as required, a detachment condition that is optimum in accordance with a criterion based on the changed settings is set as the recommended condition.

Modes from which selection is made in the mode setting window 603 are not limited to the ones listed above, and modes each giving priority to one of various items may be provided as required.

The condition display window 604 displays the recommended condition set by the recommended condition setting unit 102. The condition display window 604 includes a detailed information transition button 605. When the user presses the detailed information transition button 605, a separate window which displays information about details of the recommended condition and table information used for setting of the recommended condition is displayed. When the user presses the approval button 606, the set recommended condition is set as a condition for detaching the cell, and is stored in the storage unit 105.

Configurations and functions of the windows and the buttons illustrated in FIG. 6 are merely an example, and may be changed as required to suit functions demanded of the display unit 104. For example, the general display window 600 may further include a window that enables the user to correct the set recommended condition. To give another example, when the information processing device 10 is an information processing device used in a detachment assistance system described later, the press of the approval button 606 may cause the detachment device to operate under the recommended condition.

### Second Embodiment

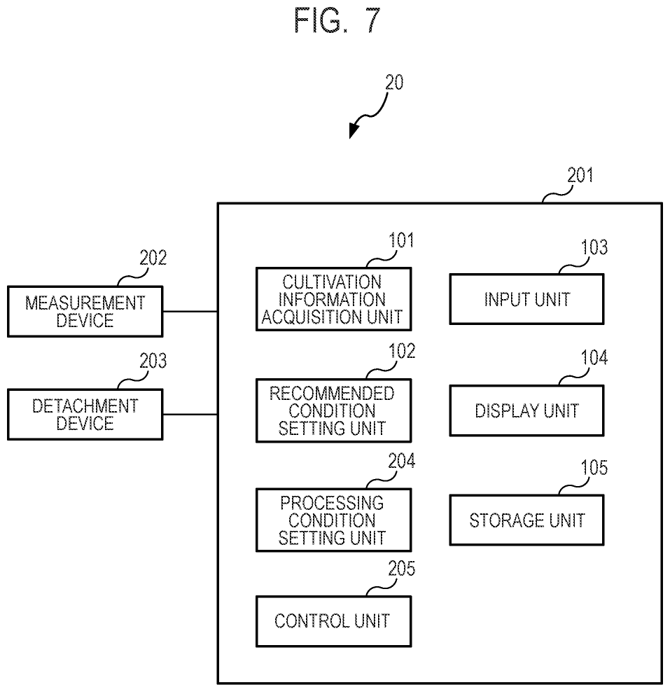

FIG. 7 is a function block diagram of a detachment assistance system 20 according to a fifth embodiment of the present invention.

The detachment assistance system 20 according to the present embodiment includes an information processing device 201, a measurement device 202, and a detachment device 203. The information processing device 201 further includes, in addition to the same components as the components included in the information processing device 10 according to the first embodiment, a processing condition setting unit 204 and a control unit 205.

The measurement device 202 is a component that executes measurement for acquisition of the cultivation information by the cultivation information acquisition unit 101. The measurement device 202 accordingly includes a vessel measurement device that executes measurement for acquisition of information about a to-be-processed vessel in which a cell has been cultured.

The detachment device 203 is a component for detachment, from the to-be-processed vessel, a cell that has been cultured in the to-be-processed vessel under a condition set with the use of the information processing device 201. The detachment device 203 may be, for example, an ultrasonic transducer.

The processing condition setting unit 204 is a component for setting a processing condition under which the detachment device 203 detaches the cell from the to-be-processed vessel. The control unit 205 is a component for controlling the detachment device 203 based on the processing condition set by the processing condition setting unit 204.

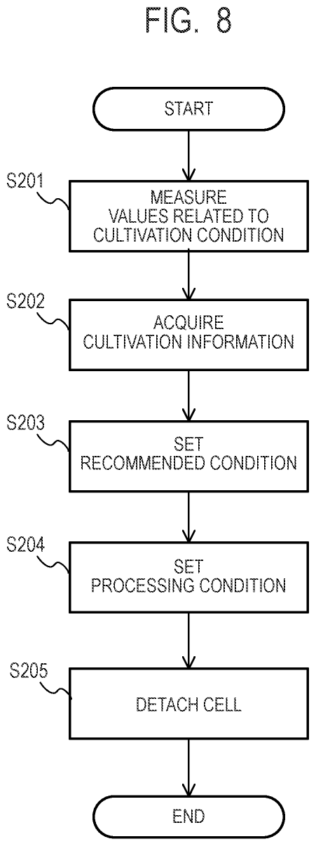

FIG. 8 is a flow chart for illustrating an outline of a cell detachment method that uses the detachment assistance system 20 according to the present embodiment.

First, in Step S201, which is a measurement step, the measurement device 202 measures values related to a cultivation condition of a cell to be detached. The measurement step of Step S201 includes a vessel measurement step in which the vessel measurement device included in the measurement device 202 executes measurement about the to-be-processed vessel.

In the subsequent Step S202, which is a cultivation information acquisition step, the cultivation information acquisition unit 101 acquires cultivation information. The cultivation information acquired by the cultivation information acquisition unit 101 includes the values measured by the measurement device 202.

In the following Step S203, which is a recommended condition setting step, the recommended condition setting unit 102 sets, based on the cultivation information acquired by the cultivation information acquisition unit 101, a recommended condition for detaching the cell from the to-be-processed vessel.

A flow from Step S202 to Step S203 is the same as a flow from Step S101 to Step S102 in the first embodiment.

In the subsequent Step S204, which is a processing condition setting step, the processing condition setting unit 204 sets a processing condition for actually executing detachment, by referring to the recommended condition set by the recommended condition setting unit 102.

The processing condition setting unit 204 may automatically perform required modification on the recommended condition, in accordance with a criterion incorporated in advance, or the recommended condition may be changed based on the user's input from the input unit 103. The processing condition setting unit 204 may set the recommended condition as the processing condition without modification when the recommended condition does not require modification.

In the next Step S205, which is a detachment step, the detachment device 203 detaches the cell from the to-be-processed vessel under control of the control unit 205. Here, the control unit 205 controls the detachment device 203 based on the processing condition set by the processing condition setting unit 204.

Various modifications can be made to the detachment assistance system 20 described in the second embodiment.

For example, the information processing device 201 included in the detachment assistance system 20 may be replaced with the information processing device 10 described in the first embodiment, and a recommended condition acquisition step of acquiring a recommended condition with the use of the information processing device 10 may be provided. Based on the acquired recommended condition, the user may manually set a processing condition for detaching the cell from the to-be-processed vessel in the processing condition setting step.

The detachment assistance system 20 may omit the processing condition setting unit 204, and the control unit 205 may control the detachment device 203 based on the recommended condition set by the recommended condition setting unit 102, to thereby detach the cell from the to-be-processed vessel.

The detachment assistance system 20 may omit the control unit 205, and the user may manually perform required control on the detachment device 203 based on the processing condition set by the processing condition setting unit 204.

To give another example, the detachment assistance system 20 may include, in addition to the components illustrated in FIG. 7, a culture device for culturing a cell. Examples of the culture device include an incubator.

According to the present invention, it is possible to provide an information processing device usable in a method of detaching a cell by applying vibration to a culture vessel, to set a cell detachment condition appropriate for a cultivation condition including the type of culture vessel used. Further, it is possible to provide a detachment assistance system, an information processing method, and a cell detachment method that use the above-mentioned information processing device. Still further, it is possible to provide a non-transitory storage medium having stored thereon an information processing program used in the above-mentioned information processing device.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While preferable embodiments are explained above, the above-mentioned example embodiments merely describe specific examples in carrying out the present invention, and are not to be construed as limiting the technical scope of the present invention in any way. That is, the present invention can be implemented in various forms without departing from the technical idea or the main features of the present invention. For example, an embodiment which is produced by adding a part of constitutions of any of the embodiments to another embodiment, and an embodiment which is produced by replacing a part of constitutions of any of the embodiments with a part of another embodiment should be understood as to be embodiments capable of being applied with the present invention.

This application claims the benefit of Japanese Patent Application No. 2022-171678, filed Oct. 26, 2022, which is hereby incorporated by reference herein in its entirety.

What is claimed is:

1. An information processing device comprising:
a cultivation information acquisition unit configured to acquire cultivation information of a cell; and
a recommended condition setting unit configured to set a recommended condition for detaching the cell from a to-be-processed vessel in which the cell is cultured,
the cultivation information including information about the to-be-processed vessel,
the recommended condition being a condition for detaching the cell by applying vibration to the to-be-processed vessel,
the recommended condition setting unit being configured to set the recommended condition based on the cultivation information, with use of information concerning an association relationship between information about a cultivation condition and information about a detachment condition,
the information about the cultivation condition including information about a culture vessel,
the information about the detachment condition including information about a condition for applying vibration to the culture vessel.

2. The information processing device according to claim 1,
wherein the information concerning the association relationship between the information about the cultivation condition and the information about the detachment condition includes a plurality of reference items based on which the recommended condition is set, and
wherein the recommended condition setting unit is configured to set the recommended condition in order of priority determined for the plurality of reference items in advance.

3. The information processing device according to claim 1, wherein the information about the culture vessel includes at least one piece of information selected from the group consisting of information about a type of the culture vessel, information about a shape of the culture vessel, information about a chemical property of the culture vessel, and information about a physical property of the culture vessel.

4. The information processing device according to claim 3, wherein the information about the type of the culture vessel includes at least one piece of information selected from the group consisting of information about a manufacturing company of the culture vessel, information about a sales company of the culture vessel, and information about a part number of the culture vessel.

5. The information processing device according to claim 3, wherein the information about the shape of the culture vessel includes at least one piece of information selected from the group consisting of information about a form of the culture vessel, information about a thickness of the culture vessel, information about a curvature of a bottom surface portion of the culture vessel, and information about a flatness and smoothness of an inner surface of the culture vessel.

6. The information processing device according to claim 3, wherein the information about the chemical property of the culture vessel includes at least one piece of information selected from the group consisting of information about a material forming the culture vessel, and information about chemical modification of an inner surface of the culture vessel.

7. The information processing device according to claim 3, wherein the information about the physical property of the culture vessel includes at least one piece of information selected from the group consisting of information about hydrophilic property of an inner surface of the culture vessel, information about a contact angle of the inner surface of the culture vessel, information about a surface charge of the inner surface of the culture vessel, and information about a natural vibration frequency of the culture vessel.

8. The information processing device according to claim 1, wherein the culture vessel includes at least one selected from the group consisting of a flask, a dish, and a well.

9. The information processing device according to claim 3,
wherein the culture vessel includes a dish, and
wherein the information about the shape of the culture vessel includes information about presence or absence of a convex portion provided in a bottom surface edge portion of the dish.

10. The information processing device according to claim 9,
wherein the convex portion is provided in the bottom surface edge portion of the dish, and
wherein the information about the shape of the culture vessel includes information about a shape of the convex portion.

11. The information processing device according to claim 1, wherein the information about the condition for applying vibration to the culture vessel includes at least one piece of information selected from the group consisting of information about an intensity of the vibration to be applied to the culture vessel, information about a frequency of the vibration to be applied to the culture vessel, and information about a time for which the vibration is applied to the culture vessel.

12. The information processing device according to claim 11,
wherein the vibration applied to the culture vessel is vibration caused by application of an ultrasonic wave that is generated by an ultrasonic transducer, and
wherein the information about the intensity of the vibration to be applied to the culture vessel includes information about a voltage to be applied to the ultrasonic transducer.

13. The information processing device according to claim 1, wherein the information about the detachment condition further includes at least one piece of information selected from the group consisting of information about a detachment fluid and information about an acoustic matching material.

14. The information processing device according to claim 1,
wherein the information about the detachment condition is information obtained based on information about a detachment result, and
wherein the information about the detachment result includes at least one piece of information selected from the group consisting of information about a detachment rate of the cell, information about a survival rate of the detached cell, information about a growth rate of the detached cell, and information about functionality of the detached cell.

15. The information processing device according to claim 1, wherein the information about the cultivation condition further includes at least one piece of information selected from the group consisting of information about the cell, information about a cell semination density, information about a culture fluid, information about a cultivation period, and information about a culture environment.

16. The information processing device according to claim 15, wherein the information about the cell includes information about cell species.

17. A detachment assistance system comprising:
a measurement device configured to execute measurement for acquisition of the cultivation information; and
the information processing device of claim 1,
wherein the measurement device includes a vessel measurement device configured to execute measurement for acquisition of the information about the to-be-processed vessel.

18. The detachment assistance system according to claim 17, further comprising a detachment device configured to detach a cell cultured in the to-be-processed vessel from the to-be-processed vessel.

19. The detachment assistance system according to claim 18, wherein the information processing device further includes a processing condition setting unit configured to set a processing condition under which the detachment device detaches the cell from the to-be-processed vessel.

20. The detachment assistance system according to claim 18, wherein the information processing device further includes a control unit configured to control the detachment device.

21. The detachment assistance system according to claim 18, wherein the detachment device includes an ultrasonic transducer.

22. A non-transitory storage medium having stored thereon an information processing program for causing a computer to:
acquire cultivation information of a cell; and
set a recommended condition for detaching the cell from a to-be-processed vessel in which the cell is cultured, the setting being executed based on the cultivation information, with use of information concerning an association relationship between information about a cultivation condition and information about a detachment condition,
the cultivation information including information about the to-be-processed vessel,
the recommended condition being a condition for detaching the cell by applying vibration to the to-be-processed vessel,
the information about the cultivation condition including information about a culture vessel,
the information about the detachment condition including information about a condition for applying vibration to the culture vessel.

23. An information processing method comprising:
a cultivation information acquisition step of acquiring cultivation information of a cell; and
a recommended condition setting step of setting a recommended condition for detaching the cell from a to-be-processed vessel in which the cell is cultured,
the cultivation information including information about the to-be-processed vessel,
the recommended condition being a condition for detaching the cell by applying vibration to the to-be-processed vessel,
the recommended condition setting step including setting the recommended condition based on the cultivation information, with use of information concerning an association relationship between information about a cultivation condition and information about a detachment condition,
the information about the cultivation condition including information about a culture vessel,
the information about the detachment condition including information about a condition for applying vibration to the culture vessel.

24. A cell detachment method comprising:
a recommended condition acquisition step of acquiring, with use of the information processing device of claim 1, the recommended condition;
a processing condition setting step of setting, based on the recommended condition, a processing condition for detaching a cell from the to-be-processed vessel; and
a detachment step of detaching the cell from the to-be-processed vessel, based on the processing condition.

25. The cell detachment method according to claim 24, further comprising a measurement step of executing measurement for acquisition of the cultivation information,
wherein the measurement step includes executing measurement for acquisition of the information about the to-be-processed vessel.

26. The cell detachment method according to claim 24, wherein the detachment step includes detaching the cell with use of an ultrasonic transducer.
